(19) United States
(12) Patent Application Publication
(10) Pub. No.: US 2005/0220715 A1
(43) Pub. Date: Oct. 6, 2005

(54) Methods and compositions for improving drug safety

(75) Inventor: Tong-Ho Lin, Taipei City (TW)

Correspondence Address: OSHA LIANG L.L.P., 1221 MCKINNEY STREET, SUITE 2800, HOUSTON, TX, 77010, US

(73) Assignee: Lotus Pharmaceutical Co., Ltd., Taipei (TW)

(21) Appl. No.: 11/093,311

(22) Filed: Mar. 30, 2005

Related U.S. Application Data
(60) Provisional application No. 60/557,573, filed on Mar. 30, 2004.

(51) Int. Cl. A61K 31/5513; A61K 49/00

(52) U.S. Cl. 424/10.1; 514/221

(57) ABSTRACT

A pharmaceutical composition with improved safety includes a selected amount of a vomit-inducing agent, wherein the selected amount is less than an amount needed to induce vomit in a user; and a therapeutic agent. The therapeutic agent may be selected from a sleeping pill, an anxiolytic, a hypnotic, a contraceptive agent. The therapeutic agent may also be selected from diazepam, flunitrazepam, alprazolam, triazolam, fludiazepam, midazolam, estazolam, zopiclone, and a combination thereof. The vomit-inducing agent may be selected from emetine, cephaeline, and a combination thereof.

### CROSS-REFERENCE TO RELATED APPLICATIONS

This application claims priority, under 35 U.S.C. § 119, of Provisional Patent Application Ser. No. 60/557,573, filed on Mar. 30, 2004. This Provisional Application is incorporated by reference in its entirety.

### STATEMENT REGARDING FEDERALLY SPONSORED RESEARCH OR DEVELOPMENT

Not applicable.

### BACKGROUND OF INVENTION

1. Field of the Invention

The invention relates generally to pharmaceutical compositions that include two or more components for improving safe use of a drug. More particularly, the present invention relates to a composition that include a therapeutic component that need to be controlled within a safe dosage to prevent overdose or abuse. Examples of such components may include sleeping pills, anxiolytics-hypnotics, and contraceptives. In addition, the present invention may also be used with agricultural chemicals or corrosive chemicals to prevent such chemicals from causing harm.

2. Background Art

Sleeping pills and anxiolytics-hypnotic medications are for treating depression, anxiety, or related disorder. Although these drugs can relax muscles, alleviate anxiety, and induce sleep, they can be addictive. Therefore, such medications should not be taken for long term without proper supervision; particularly, users should not increase the dosage on their own.

Overdose of sleeping pills or anxiolytics-hypnotics can lead to accident or death. The benzodiazepine-containing anxiolytics-hypnotics or barbital-containing sleeping pills or anti-anxiety pills can become addictive. In addition, short-lasting benzodiazepine-containing sleeping pills (e.g., triazolam) and long-lasting benzodiazepine-containing sleeping pills (e.g., diazepam) may be used as date-rape drugs to spice drinks to make people unconscious.

Furthermore, highly toxic agricultural chemicals, such as parathion, or corrosive chemicals, such as sulfuric acid or hydrochloric acid, may be used to commit suicide or to cause injuries to others.

Disease or psychological/physiological impairment that are cause by chemical, physical, bacterial, or viral factors often require treatment with pharmaceutical compositions. Proper use of such pharmaceuticals may rely on professional or common sense knowledge. The prevalent drug abuse in the modern society not only impacts individual lives, but also adversely affect the society in every aspect. Whether a drug is properly used depends on the considerations of therapeutic efficacy, dosage, and disease conditions. Therefore, it often requires diagnosis, observation, and judgment of medical professionals. If patients arbitrarily change drug uses based on their body conditions or their convenience, it may cause all sorts of undesired side effects and lead to irreparable damages. Some medications, which may be effective for similar illness, when improperly used (e.g., at different dosage) may cause severe side effects. The absorption and therapeutic effects of drugs are related to their mechanisms of action. Patients do not have the knowledge required and should not base on incomplete information to arbitrarily adjust dosage to “cure” symptoms. Drug safety is an integral part of medical treatment. How to accomplish safe drug use requires coordination of various factors.

A typical example of a drug that might be misused or abused is mifepristone, which is a steroid hormone compound. In 1989, E. E. Baulieu disclosed reported that mifepristone has high affinity for the progesterone receptors and could block the functions of corticoids. See, Science, vol. 245, No. 4924, pp. 1351-1357. Because progesterone is essential for pregnancy, occupation of the progesterone receptors by mifepristone prevents the binding of progesterone to its receptor, leading to interruption of progesterone function, uterus contraction, and shedding of uterine lining. As a result, pregnancy is aborted.

Based on this observation, the French pharmaceutical company, Roussel-Uclaf (Romainville, France), developed mifepristone (RU-486), which can terminate early pregnancy, prevent embryo attachment, induce period, and induce cervix maturation. The chemical structure of mifepristone (RU-486) is shown as formula (I): 17β-hydroxy-11β-(4-dimethylaminophenyl)-17α-(1-propynyl)-estra-4,9-dien-3-one.

In 1993, R. Peyron et al. reported that within the first 49 days of pregnancy, administration of 600 mg RU-486 and 400 mg misprostol can lead to early termination of the pregnancy with a success rate of 97%. See, N. Engl. J. Med., Vol. 328, pp. 1509-1513. RU-486 currently is available in China (since 1988), France (since 1990), England (since 1991), Sweden (since 1992), and the United States (since 2000). More recently, Taiwan (Dec. 28, 2000) also approved RU-486 for sale.

During normal menstrual cycles, administration of RU-486 at different stages produces substantially different effects. There are four phases of a menstrual cycle: menstruation, the follicular phase, ovulation, and the luteal phase. If RU-486 is administered during early luteal phase, the copus leuteum will degrade. If RU-486 is given during mid luteal phase, the concentrations of luteinizing hormone and estrogen increase, leading to bleeding. This bleeding is in addition to the normal menstruation. If RU-486 is give in late luteal phase, it will induce bleeding and shorten the luteal phase and prolongs the follicular phase. If RU-486 is given within 3 days prior to menstruation, it has no impact on the cycle. If RU-486 is given late in the follicular phase, it will prolong the follicular phase by 10-15 days as a result of the induction of luteinizing hormone release. Thus, RU-486 hs various effects depending on the time of administration.

The doses need for artificial abortion using RU-486 can vary quite a bit. Although R. Peyron et al. suggested 600 mg, which became the most adopted dosage, later research has shown that this dosage can be flexibly adjusted. For example, Anonym et al. reported using 200 mg of RU-486 (Acta Obstet. Gynecol. Scand. Vol. 80, pp. 447-451, 2001), and B. Xiao et al. reported using 150 mg of RU-486 (Contraception, vol. 68, No. 6, pp. 489-494, 2003). Both showed good results with abortion. WHO also reported that for morning after pregnancy prevention, RU-486 at 50 mg or 10 mg can produce the same effect as 500 mg. See, Lancet, vol. 353, No. 9154, pp. 697-702, 1999. H. V. Hertzen (Lancet, vol. 360, pp. 1803-1810, 2002) and H. Hamoda (Obstet. Gynecol., vol. 104, No. 6, pp. 1307-1313, 2004) also reported that 10 mg RU-486 could produce the emergency contraceptive effects.

The above examples illustrate that various medications can be used in different situations for different purposes. This knowledge is beyond the reach of average patients. Therefore, it is desirable that pharmaceutical compositions be prepared in a form that is designed for specific purposes and cannot be easily abused.

### SUMMARY OF INVENTION

One aspect of the invention discloses methods and compositions for improving the safety of drugs. A composition in accordance with one embodiment of the invention contains a suitable amount of vomit-inducing agent and a low dose of the main component (therapeutic component), such as sleeping pills, anxiolytics-hypnotics, and contraceptives. Such compositions can prevent overdose of the main ingredient. This approach can also be used to prevent the ingestion of agriculture chemicals or corrosive chemicals, for example.

Another aspect of the invention relates to methods for producing a composition having a vomit-inducing agent and a main component, such as sleeping pills, anxiolytics-hypnotics, contraceptives. The methods can also be used with agriculture chemicals and corrosive chemicals. A method in accordance with one embodiment of the invention can provide a product having a vomit-inducing agent in a controlled amount that can be released before the main components are released, in order to promote the safe use of the drugs.

Other aspects and advantages of the invention will be apparent from the following description and the appended claims. One of ordinary skill in the art, having read the following description and the associated drawings and examples, would appreciate the purposes and advantages of the present invention.

### BRIEF DESCRIPTION OF DRAWINGS

FIG. 1 shows chemical structures of mifepristone (RU-486) and emetine analogs.

FIG. 2 shows pharmacokinetics of a mifepristone composition in accordance with one embodiment of the invention.

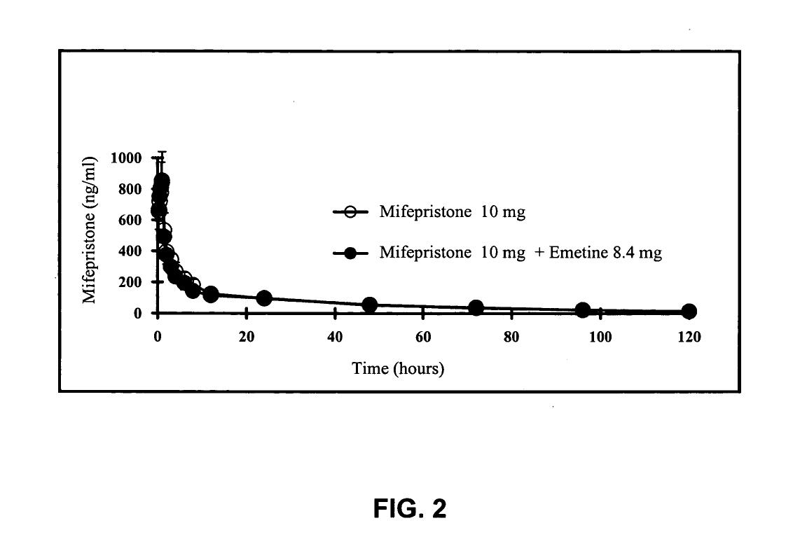

### DETAILED DESCRIPTION

Embodiments of the invention relate to compositions for improving drug safety and methods for producing such compositions. A composition in accordance with embodiments of the invention can prevent drug abuse and improve drug safety. In addition, such compositions can also prevent ingestion of agriculture chemicals or corrosive chemicals. Such compositions can also prevent intentional overdose of sleeping pills, anxiolytics-hypnotics, contraceptives, and the like, that may lead to permanent injuries or undesired side effects.

The present invention discloses compositions containing suitable amounts of vomit-inducing agents and safe doses of main components, such as therapeutic agents. When such compositions are ingested, the vomit-inducing agents may reach a concentration that will irritate the GI system to induce nausea and/or vomiting before the concentrations of the main components (e.g., active ingredients) reach too high to cause injuries. Some embodiments of the invention include suitable amounts of vomit-inducing agents in low amounts of agriculture chemicals or corrosive chemicals to prevent ingestion of such chemicals. The vomit-inducing agents will cause nausea and/or vomiting and prevent such chemicals form causing significant damages to whoever ingests them.

If the vomit-inducing agents are to be added to a pharmaceutical composition, such as benzodiazepine anxiolytics-hypnotics or hydrochloride salts of barbitals (sleeping pills), they may be added while formulating the compositions. In this case, their concentration should adjusted to allow the main ingredients to have their normal effects (sedation or sleep inducing) before the vomit-inducing agents accumulate to their effective concentrations. Alternatively, these vomit-inducing agents may be added to the main components first before formulation. Addition of vomit-inducing agents in accordance with embodiments of the present invention is to improve the safety of normal drug use.

Among the above-described benzodiazepine sleeping pills or anxiolytics-hypnotics, the long-lasting diazepam, moderately long-lasting flunitrazepam, short-lasting alprazolam, ultra-short-lasting triazolam, or fludiazepam, midazolam, estazolam, and non-benzodiazepine (zopiclone) are all prescription drugs. Because of the abuse of these drugs, these are now listed as controlled substance. The agricultural chemicals referred to in this description include parathion, parathion-methyl, folidol, methanmidophos, mepanipyrim, mecarbam. The corrosive chemicals referred to in this description include sulfuric acid and hydrochloric acid. Vomit-inducing agents (compounds) may include any known in the art, such as ipeace, emetine, cephaeline, or mixtures thereof.

One embodiment of the invention comprises a suitable amount of a vomit-inducing agent and a low dose of a pharmaceutical, such as RU-486. This composition may prevent overdose of RU-486. As noted above, RU-486, a progesterone receptor antagonist, can compete with progesterone for binding to the progesterone receptors, leading to antagonism of the effects of progesterone on uterus. Therefore, administration of RU-486 during pregnancy can cause uterine muscle to become sensitive to prostaglandin-induced uterus contraction. Accordingly, RU-486 can terminate early pregnancy, prevent embryo attachment to the uterus, induce menstruation, and stimulate cervix maturation, leading to medical abortion.

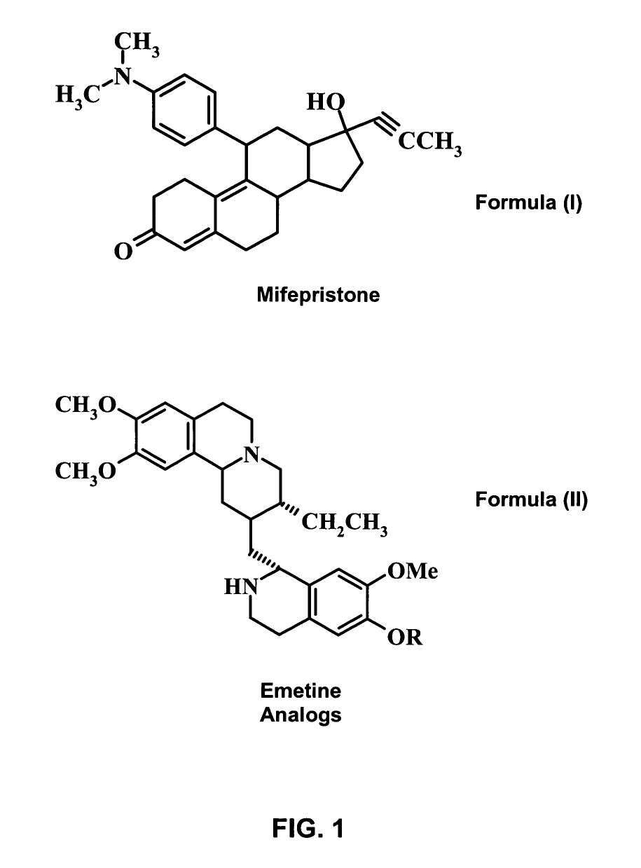

One of the vomit-inducing agent that may be used with embodiments of the invention is ipeace. Ipeace is a herbal medication from dry stems or roots of Cephaelis ipecacuanha or Cephaelis acuminate. According Chinese Pharmacopoeia, ipeace contains 2.0% or more alkaloids. The alkaloids in ipeace include emetine (6′,7′,10,11-tetramethoxyemetean) and cephaeline (desmethylemetine), having a structure shown in FIG. 1 as Formula (II). It is known that ipeace from different sources have different ratios of emetine to cephaeline. Quang and Woolf reported that C. ipecacuanha contains ⅓ emetine and ⅔ cephaeline, while C. acuminate has an equal amount of emetine and cephaeline. See, Curr. Opin. Pediatr., vol. 12, No. 2, pp. 153-162. As a result, Pharmacopoeia from different countries list ipeace as having different alkaloid compositions. For example, British Pharmacopoeia lists ipeace as having 72% emetine and 26% cephaeline, while U.S. Pharmacopoeia (“USP”) lists ipeace as having a ratio of emetine to cephaeline of 1:2.5.

Ipeace can induce nausea and vomiting because emetine and cepaheline directly stimulate gastric intestine tract. Commonly used ipeace preparations include emetine pastes and emetine syrups. Emetine pastes typically are about 14 times the potency of emetine syrups. USP (DI 1997) lists emetine syrups for adolescence and adult use at a dose of 15-30 ml mixed in 240 ml water for oral use. If after 20-30 minutes no vomit is induced, repeat with the same dose. In 85% of patients, vomits are induced in about 20-30 minutes. After second dose, 95% of patients vomit. Emetine preparations have been in use for 30-40 years. Therefore, they have been shown to be very safe.

Pharmaceutical compositions in accordance with embodiments of the invention may include, for example, sleeping pills, anxiolytics-hypnotics, or contraceptives that include a suitable dose of active ingredients and suitable amount of a vomit-inducing agent (such as emetine). When a user takes one or two pills (or the intended number of pills), the desired sleep-inducing, anxiolytic-hypnotic, or contraceptive effects are realized. However, when a user takes more than the intended dosage, the vomit-inducing agents may accumulate to an extent to induce vomit, which then forces out all compositions (including the sleep-inducing, anxiolytic-hypnotic, and contraceptive ingredients). As a result, the body cannot accumulate the pharmaceutical compositions to a dangerous extent and, therefore, it can avoid drug overdose or abuse. In the above described example, the pills are designed to have the desired sleeping, anxiolytics-hypnotic, or contraceptive effects with 1 or 2 pills. If a user intends to commit suicide or induce medical abortion by taking a large number of pills, the cumulative amount of vomit-inducing agent in these pills will induce vomit and force all compositions of the pills out. The user will not be able to ingest enough amount to result in death or unsupervised medical abortion.

In accordance with embodiments of the invention, the above described compositions can prevent drugs from being used to spike drinks as date rape drugs. Because the vomit-inducing agent will force the ingredients out before sufficient sleeping or anxiolytic-hypnotic ingredients are absorbed. Therefore, the major ingredient will not accumulate enough to make someone unconscious.

In accordance with embodiments of the invention, low dose sleeping pills, anxiolytic-hypnotic pills, and contraceptive pills can still have their intended physiological functions—inducing sleepiness, sedation, and contraception. For example, low dose RU-486 can still be used as contraceptive, but cannot be used to terminate pregnancy. RU-486 when used at high dose can terminate pregnancy. In order to prevent users from inducing medical abortion without physician supervision or prescription, leading to bodily injuries or death, compositions of the invention include vomit-inducing agent and a low dose of an active ingredient. These compositions have practical utility.

Compositions of the invention include suitable amounts of vomit-inducing agents and a low dose active ingredient, such as sleeping pills or anxiolytic-hypnotic ingredients. The vomit-inducing agent for example may be emetine. The concentrations of emetine in such compositions, for example, may be in a range from about 0.2 mg to about 300 mg, while the sleeping pills or anxiolytic-hypnotic agent may be in a range from about 0.02 mg to about 250 mg, depending on specific components. The compositions may also include pharmaceutically acceptable carriers or additives. Such pharmaceutically acceptable carriers and additives are well known in the art. In preferred embodiments, a composition may include emetine in an amount from about 2.1 mg to about 29.4 mg and an active ingredient (e.g., sleeping or anxiolytic-hypnotic ingredient) in an amount from about 5 mg to about 100 mg. In another embodiment, the composition may include cephaeline in an amount from about 2.1 mg to about 100 mg and an active ingredient (e.g., sleeping or anxiolytic-hypnotic ingredient) in an amount from about 5 mg to about 100 mg. In more preferred embodiments, a composition of the invention may include emetine in an amount from about 4.2 mg to about 21 mg and an active ingredient (e.g., sleeping or anxiolytic-hypnotic ingredient) in an amount from about 10 mg to about 100 mg. Alternatively, a composition may include cephaeline in an amount from about 4.2 mg to about 63 mg and an active ingredient (e.g., sleeping or anxiolytic-hypnotic ingredient) in an amount from about 5 mg to about 100 mg.

In embodiments of the invention that include RU-486, the vomit-inducing agent may be emetine in an amount from about 0.25 mg to about 42 mg or cephaeline in an amount from about 0.25 mg to about 126 mg, while the RU-486 may be in an amount from about 0.25 mg to about 150 mg. The compositions may also include pharmaceutically acceptable carriers or additives. In preferred embodiments, the vomit-inducing agent may be emetine in an amount from about 2.1 mg to about 29.4 mg or cephaeline in an amount from about 2.1 mg to about 120 mg, while the RU-486 may be in an amount from about 5 mg to about 100 mg. In more preferred embodiments, the vomit-inducing agent may be emetine in an amount from about 4.2 mg to about 21 mg or cephaeline in an amount from about 4.2 mg to about 63 mg, while the RU-486 may be in an amount from about 10 mg to about 100 mg.

If necessary, compositions of the invention may include various additives, carriers, or diluents. Such additives, carriers, and diluents are well known in the art. They can be prepared as liquid formulations or patches or paste for direct application on the skin. Such preparations may be manufactured using any methods known in the art. For example, starch, carboxymethyl cellulose (CMC), or similar binders may be added to prepare the compositions into tablets, pills, or capsules. Slow release additives may also be added to these preparations to make slow release tablets or capsules using methods known in the art.

In accordance with some methods for preparation of compositions of the invention, emetine and the active ingredient (e.g., RU-486) may be prepared as separate granules, which may be pressed into double-layered tablets or granules, having different rates of release. Alternatively, different coating technologies may be used to coat different granules with fast release coating, slow release coating, acid-resistant coating, or the like. In accordance with some embodiments of the invention, various coating techniques may be used to coat the vomit-inducing agent (e.g., emetine or cephaeline) such that it will be released before (e.g., 10 to 30 minutes before) the release of the active ingredient (e.g., RU-486).

some embodiments of the invention relate to compositions having vomit-inducing agents and low dose RU-486 in a compound formulation, having a core tablet portion and a film-coated outer tablet. Each of these two portions may be coated with different coating techniques. For example, these techniques may judicially use acid-resistant coatings, fast release coatings, or slow release coatings in combination so that the vomit-inducing agent will be released to an effective concentration to induce vomit before the active ingredient (e.g., RU-486) is release to a high concentration.

Composite composition preparations of the invention may use acid-resistant coating and fast release coating in combination, for example. Methods for such preparation may use microcrystalline cellulose and some sodium starch glycolate to mix with the active ingredient (such as the sleeping, anxiolytic-hypnotic, or contraceptive ingredient). Then, polyvynylpyrrolidone (Povidone) K-30 is added to prepare granules. Finally, sodium starch glycolate and magnesium stearate are mixed in. The resultant granules are then pressed to produce core tablets. The core tablets are first coated with a base coating. The vomit-inducing agent (e.g., emetine or cephaeline) are mixed with polyethylene glycol (PEG), titanium oxide (TiO2), or the like to make a coating solution. Then, this coating solution is used to further coat the core tablets that have been previously coated with the base coating.

Some embodiments of the invention can be prepared with capsule preparation techniques. First, the active ingredient (e.g., the sleeping, anxiolytic-hypnotic, or contraceptive ingredient) is prepared as core pellets, which are then coated with an acid-resistant base coating. The vomit-inducing agent and other compounds (e.g., PEG, TiO2) are made into a coating solution as described above. This coating solution is then used to make a second coating on the core pellets. The double coated pellets can then be put on capsule filling machines to produce the capsules.

### EXAMPLES

To illustrate the present invention. The following three experiments were carried out: (1) clinical trials of RU-486 and emetine compositions in inducing vomit; (2) blood concentrations of emetine from the RU-486 and emetine composition; and (3) pharmacokinetic studies in animals using the RU-486 and emetine composition.

Based on the results from the first to fifth groups of vomit-inducing experiments (described below), single oral dose of the composition having 16.8 mg emetine (e.g., Group 2) induces 0% vomit. If the composition contains 42 mg emetine (Group 1 or 3), then vomit is induced in 80%-90% of the subjects. If the composition contains 50.4 mg emetine (Group 4 or 5), then vomit is induced in 90%-100% of the subjects.

In addition, the first group in Experiment 1, which was monitored for the rates of vomit induction for two hours after oral administration of the composition, was also monitored for blood concentrations of emetine 30 minutes after oral administration. Among the 10 healthy, female test subjects, eight vomited (80% induction rates). The two test subjects who did not vomit have blood emetine concentrations of 2.8 ng/ml and 1.9 ng/ml, which are lower than the blood emetine concentrations of the other eight test subjects who vomited.

The purpose of the clinical test is to show that if a user takes a quantity of RU-486 and emetine composition that would have been sufficient to terminate pregnancy (instead of contraception), then emetine will induce vomit to prevent sufficient RU-486 to be absorbed to terminate the pregnancy. The purpose of the pharmacokinetic experiment is to show that the composite composition of RU-486 and emetine would not have a different absorption rate for RU-486 because of the presence of emetine, and hence, emetine and RU-486 do not interfere with each other's activity. If RU-486 is absorbed at a different rate, it may change the contraceptive efficacy.

Experiment 1: Clinical Trials of Vomit Induction by Compositions Containing RU-486 and Emetine

This experiment includes five groups in clinical tests, depending on the prescription and dosages. The test subjects first report their health conditions for the past week. Those had periods were excluded. The test subjects were then informed about drug safety. After administration of the test compositions, the test subjects were observed for their emotional states. If there was any change in their emotional states, medical professionals were notified. In each test group, ten healthy female subjects were selected based on their health conditions, excluding those having periods.

Single oral doses of the test drugs were administered to the test subjects before meals. After two hours, the vomiting responses were monitored. The prescription, doses, and protocols are described in detail below.

The first group included ten healthy female test subjects. Each was given a single oral dose of five composite tablets (Preparation I, described below), each of which includes 10 mg RU-486 and 8.4 mg emetine.

The second group included ten healthy female test subjects. Each was given a single oral dose of two since tablets (Preparation I, described below), each of which includes 10 mg RU-486 and 8.4 mg emetine.

The third group included ten healthy female test subjects. Each was given a single oral dose of 10 since tablets (Preparation II, described below), each of which includes 10 mg RU-486 and 4.2 mg emetine.

The fourth group included ten healthy female test subjects. Each was given a single oral dose of four since tablets (Preparation III, described below), each of which includes 10 mg RU-486 and 12.6 mg emetine.

The fifth group included ten healthy female test subjects. Each was given a single oral dose of three since tablets (Preparation IV, described below), each of which includes 10 mg RU-486 and 16.8 mg emetine.

Experiment 2: Emetine Blood Concentration Determinations

Blood concentrations of emetine in the first group of test subjects described above was also determined. In addition to monitoring the test subjects for incidents of vomiting for 2 hours after administration of the pills, blood samples (10 ml each) from the test subjects were also collected at 30 minutes after oral administration of the pills.

Experiment 3: Methods for Determining Blood Concentrations of Emetine

Blood concentrations of emetine were determined using high performance liquid chromatography (HPLC). The conditions for the HPLC analysis were as follows:

| | |
|---|---|
| Column: | Waters Symmetry Shield RP18, |
| | 5 μm, 3.9 × 150 mm; |
| Mobile Phase: | Methanol: 25 mM Na₂HPO₄ |
| | (pH = 8) = 70:30 (v/v); |
| Pump flow rate: | 0.9 ml/min; |
| Detector: | fluorescence detector, excitation |
| | 285 nm, emission 316 nm; |
| Sample Injection Volume: | 50 μL. |

Plasma sample preparation: 2 ml plasma sample was placed in a 15 ml centrifuge tube. Procainamide (4000 ng, dissolved in methanol) was added as an internal standard. The sample was thoroughly mixed. A solution of NaOH (50 μl, 0.3M) was added to the sample to raise the pH to 9.0, followed by 7 ml of ethyl acetate. The mixture was mixed for 5 minutes by rotation and then centrifuged at 3,000 rpm for 5 minutes. The upper clear solution was collected, to which 200 μl HCl (0.01M) was added. The mixture was mixed for 5 minutes by rotation. Then, the layers were separated by centrifugation at 3,000 rpm for 5 minutes. The lower layer (aqueous layer) was collected. Each HPLC injection used 50 μl of sample.

Experiment 4: RU-486 and Emetine Composition Pharmacokinetics Studies

The studies were carried out with two test groups in parallel. The first group used the composite tablets according to preparation I described below (10 mg RU-486 and 8.4 mg emetine). The control group used tablets containing only 10 mg RU-486. The two types of tablets were similarly prepared, and they had the same appearance. A total of sixteen test subjects were involved, eight in each group. Each test subject was given one pill and blood samples were collected at 0, 0.33, 0.5, 0.75, 1, 1.5, 2, 3, 4, 6, 8, 12, 24, 48, 72, 96, and 120 hours for the determination of RU-486 concentrations in order to calculate Cₘₐₓ, Tₘₐₓ, AUC₀₋₁₂₀, AUC_{0-∞}, and T_{1/2} pharmacokinetics parameters.

Experiment 5: Analysis of Pharmacokinetics Samples

RU-486 blood concentration determinations: The analysis used calibrated HPLC. Calibration was performed for a concentration range from 5 to 5000 ng/ml. The internal standard for the analysis was metoclopramide, at 20 ng/ml, 400 ng/ml, and 4000 ng/ml as quality control. The lowest detectable concentration (LOQ) is 5 ng/ml.

| | |
|---|---|
| Column: | LiChrospher ®, 5 μm, 4 × |
| | 250 mm, Merck KGaA; |
| Column Temperature: | 40° C.; |
| Mobile Phase: | 40% CH₃CN + 0.2% H₃PO₄; |
| Pump flow rate: | 0.7 ml/min; |
| Detection: | ultraviolet light, 306 nm; |
| Sample Injection Volume: | 30 μL. |

Plasma sample preparation: Plasma (0.2 ml) was placed in a test tube, to which metoclopramide (25 μL; 7.5 ng/μL) was added as internal standard, followed by 50 μL of 0.2 M K₂HPO₄. The mixture was mixed by shaking for 30 seconds. Then, 3 ml of tert-butyl methyl ether was added as the extraction reagent. The mixture was mixed by shaking for 60 seconds before centrifuged at 3,000 rpm for 5 minutes. The upper clear solution was removed and placed into another test tube. This clear solution was dry with a stream of N₂. To the dried residue, 200 μL of mobile phase was added. The tube was shaken for 60 seconds. Each injection used 30 μL of sample.

Results from the Vomit Induction by RU-486 and Emetine Compositions

Each group in Test 1 has 10 healthy female test subjects. Each test subject was given oral dose of a composite tablet (see below) before meal. During the two hours following drug administration, the subjects were monitored for vomiting responses. Results from these tests are as follows:

The first group showed that eight out of 10 test subjects had vomiting responses. Thus, the vomit induction rate was 80% for the first group. The second group showed none of the 10 test subjects had any vomit response. Thus, the vomit induction rate was 0% for the second group. In the third group, 9 test subjects vomited. Thus, the vomit induction rate was 90% in the third group. The fourth group also had a 90% induction rate. Everyone in the fifth group had a vomiting response. Thus, the vomit induction rate was 100% in the fifth group.

Blood Emetine Concentrations from Administration of RU-486 and Emetine Compositions.

The first group test subjects (10 subjects) were given five pills prepared according to Preparation I (described below) and monitored for 2 hours for vomiting responses. In addition, blood samples were collected from these test subjects at 30 minutes post drug administration. The blood concentrations of emetine in these 10 subjects were: 2.8, 7.7, 8.8, 9.6, 10.2, 1.9, 7.6, 7.8, 7.6, and 12.1 ng/ml. Thus, the average blood emetine concentration was 7.6±3.1 ng/ml.

RU-486 and Emetine Composition Pharmacokinetics Studies in Animals

Animal pharmacokinetic parameter calculations and statistics are shown in the following table 1.

TABLE 1

| Pharmacokinetics | Preparation I | |
|---|---|---|
| Parameters | Composite Tablet | Mifepristone |
| AUC₀₋₁₂₀ (ng/ml × hr) | 8306.15 ± 1290.59 | 8715.05 ± 1474.70 |
| AUC_{0-∞} (ng/ml × hr) | 8945.05 ± 1362.87 | 9422.92 ± 1547.96 |
| Tₘₐₓ (hr) | 0.88 ± 0.13 | 0.91 ± 0.19 |
| T_{1/2} (hr) | 33.81 ± 2.02 | 35.24 ± 2.86 |

Based on statistic results, the Preparation I composite tablets and RU-486 tables (10 mg) do not have significant difference in their pharmacokinetic parameters, such as Cₘₐₓ, Tₘₐₓ, AUC₀₋₁₂₀, AUC_{0-∞}, and T_{1/2}. The values of these parameters at 90% confidence level are as follows: ln Cₘₐₓ=87.78%-123.87%, ln AUC₁₋₁₂₀=82.93%-109.96%, and ln AUC_{0-∞}=82.87%-109.10%. Based on these results, RU-486 and emetine composition has the same biological activity as RU-486 alone.

Preparation I: Composite Tablets (RU-486 10 mg and Emetine 4.2 mg).

This embodiment provides a composition with a fast release film (coating) containing a vomit-inducing agent and an active ingredient core tablet. This composition includes the following:

(1) An active ingredient releasing core tablet comprises:

| | mg/Core Tablet |
|---|---|
| Mifepristone | 10.0 |
| Lactose | 74.5 |
| Microcrystalline cellulose 102 | 10.0 |
| Corn Starch (dry) | 15.0 |
| Povidone K-30 | 3.6 |
| Sodium Starch Glycolate | 4.0 |
| Magnesium Stearate | 0.9 |
| Tablet Core weight range | 120 ± 5% |

(2) An Acid-Resistant Film-Coating Includes:

| | mg/Tablet |
|---|---|
| Ipecacuanha Alkaloid | 4.2 |
| HPMC | 4.2 |
| OEG 6000 | 1.2 |
| TiO2 | 1.2 |
| Lake | 0.4 |
| Coating Weight Range | 11.2 |

The above two components were used to produce composite tablets (Preparation I tablets) according to the following methods:

I. Core Tablets

A. Methods for manufacturing:
1. Mix mifepristone, microcrystalline cellulose 102, and part of sodium starch glycolate. Then, lactose and corn starch dry are added and mixed in a high speed mixer for 10 minutes.
2. Dissolve Povidone K-30 in a suitable amount of water. Use a mixer to help achieve complete dissolution.
3. Slowly mix the solution from step 2 into the solution prepared in step 1. The resultant mixture was quickly poured into a mixer to prepare granules according to the wet granule preparation procedures known in the art.
4. Collect the granules and allow them to dry in a dryer tray in a room with good air circulation. The drying was performed at 45° C. until the water content was about 1-3%. These dry granules were then screened for proper sizes.
5. Screen the dry granules using a screen with 1 mm openings in a double axis granule screening machine.
6. After screening, the granules were poured into a cone-shaped mixer. To the granules, part of the sodium starch glycolate, magnesium stearate was mixed in for 15 minutes.
7. Use a reciprocating tablet machine to produce the tablets, and at the same time, perform in-process control tests.

Film Coating:

A. Under coating preparation and the first coating:
1. Dissolve Tween 80 and PEG 6000 in purified water, followed by addition of Talc. The resultant mixture was thoroughly mixed. Finally, Eudragit L-30D was added, and the mixture was thoroughly mixed until homogeneous.
2. Put the core tablets in the container having the coating solution. Coat the core tablets with the base coating solution. When the weight of the core tablet increases 0.5%-3%, the coating is complete.

B. Preparation of Ipecacuanha coating and the second coating:
1. Add HPMC, PEG 6000, TiO2, and Lake into Ipecacuanha alkaloid and mix the mixture thoroughly.

2. Add the core tablets having the first coating (base coating, described above) to the second coating solution to prepared the second coat containing Ipecacuanha.

Preparation II: Composite Tablets (RU-486 10 mg and Emetine 2.1 mg

This embodiment provides a composition with a fast release film (coating) containing a vomit-inducing agent and an active ingredient core tablet. This composition includes the following:

(1) An active ingredient releasing core tablet comprises:

| | mg/Core Tablet |
|---|---|
| Mifepristone | 10.0 |
| Lactose | 74.5 |
| Microcrystalline cellulose 102 | 10.0 |
| Corn Starch (dry) | 15.0 |
| Povidone K-30 | 3.6 |
| Sodium Starch Glycolate | 4.0 |
| Magnesium Stearate | 0.9 |
| Tablet Core weight range | 120 ± 5% |

(2) An Acid-Resistant Film-Coating Includes:

| | mg/Tablet |
|---|---|
| Eudragit L-30D | 0.28-1.69 |
| HPMC | 2.1 |
| OEG 6000 | 0.6 |
| TiO2 | 0.6 |
| Lake | 0.2 |
| Coating Weight Range | 5.6 |

The preparation composition as described above may be used to prepare composite tablet 2 (preparation II tablets), using methods describe above for the preparation of Preparation I tablets.

Preparation III: Composite Tablets (Ru-486 10 mg and Emetine 6.3 mg)

This embodiment provides a composition with a fast release film (coating) containing a vomit-inducing agent and an active ingredient core tablet. This composition includes the following:

(1) An active ingredient releasing core tablet comprises:

| | mg/Core Tablet |
|---|---|
| Mifepristone | 10.0 |
| Lactose | 74.5 |
| Microcrystalline cellulose 102 | 10.0 |
| Corn Starch (dry) | 15.0 |
| Povidone K-30 | 3.6 |
| Sodium Starch Glycolate | 4.0 |
| Magnesium Stearate | 0.9 |
| Tablet Core weight range | 120 ± 5% |

(2) An Acid-Resistant Film-Coating Includes:

| | mg/Tablet |
|---|---|
| Eudragit L-30D | 0.28-1.69 |
| PEG 6000 | 0.009-0.051 |
| Tween 80 | 0.005-0.026 |
| Talc | 0.027-0.152 |
| Purified Water | 0.028-1.69 |
| Coating Weight Range | 0.6-3.6 |

(3) Another active ingredient fast release coating substance Includes:

| | mg/Tablet |
|---|---|
| Ipecacuanha alkaloid | 6.3 |
| HPMC | 6.3 |
| PEG 6000 | 1.8 |
| TiO2 | 1.8 |
| Lake | 0.6 |
| Coating Weight Range | 16.8 |

Preparation IV: Composite Tablets (RU-486 10 mg and Emetine 8.4 mg)

This embodiment provides a composition with a fast release film (coating) containing a vomit-inducing agent and an active ingredient core tablet. This composition includes the following:

(1) An active ingredient releasing core tablet comprises:

| | mg/Core Tablet |
|---|---|
| Mifepristone | 10.0 |
| Lactose | 74.5 |
| Microcrystalline cellulose 102 | 10.0 |
| Corn Starch (dry) | 15.0 |
| Povidone K-30 | 3.6 |
| Sodium Starch Glycolate | 4.0 |
| Magnesium Stearate | 0.9 |
| Tablet Core weight range | 120 ± 5% |

(2) An Acid-Resistant Film-Coating Includes:

| | mg/Tablet |
|---|---|
| Eudragit L-30D | 0.28-1.69 |
| PEG 6000 | 0.009-0.051 |
| Tween 80 | 0.005-0.026 |
| Talc | 0.027-0.152 |
| Purified Water | 0.028-1.69 |
| Coating Weight Range | 0.6-3.6 |

(3) Another active ingredient fast release coating substance Includes:

| | mg/Tablet |
|---|---|
| Ipecacuanha alkaloid | 8.4 |
| HPMC | 8.4 |
| PEG 6000 | 2.4 |
| TiO2 | 2.4 |
| Lake | 0.8 |
| Coating Weight Range | 22.4 |

Preparation V: Composite Tablets (RU-486 10 mg and Emetine 2.1 mg)

This embodiment provides multi-layer active ingredient pellet compositions as follows:

(1) An Active Ingredient Releasing Core Pellet Comprises:

| | mg/Core Tablet |
|---|---|
| Mifepristone | 10.0 |
| Sugar Sphere #20-25 | 268 |
| Povidone K-30 | 1.5 |
| Alcohol | 0.05 |
| Pellet Core weight range | 279.5 |

(2) An Acid-Resistant Base Film-Coating Includes:

| | mg/Tablet |
|---|---|
| Eudragit L-30D | 0.84-5.07 |
| Triethyl Citrate | 0.084-0.057 |
| Purified Water | 0.0025-0.0151 mL |
| Base Coating Weight Range | 0.924-5.577 |

(3) Another Active Ingredient Fast Release Coating Substance Includes:

| | mg/Tablet |
|---|---|
| Ipecacuanha alkaloid | 2.1-8.4 |
| Povidone K-30 | 0.63-2.52 |
| Talc | 1.05-4.2 |
| Lake | 0.4-1.6 |
| Coating Weight Range | 4.18-16.72 |

The above multi-layer coatings pellets weight about 285 mg-303 mg. Fill No. 2 capsules with these pellets to produce the pellet capsules.

The above compositions may also be prepared as gel caps according to the following procedures:

III. Core Tablets:

I. Core Pellets: Active Ingredient Mifepristone Coating
1. Dissolve Mifepristone and Povidone in Alcohol. Thoroughly stir it until complete dissolution to produce solution A.
2. Put sugar spheres in a spray coating machine (GEA Strea-1). The temperature is set at 45° C. Pre-warm the system for 5 to 10 minutes. Control the blowing rate so that the pellets are evenly rolled and mixed.
3. Place the siphon tube of the sprayer into solution A. Start the mixer and the sprayer pump to start active ingredient (mifepristone) pellet spray coating.

II. Second Layer Acid-Resistant Ingredient Base Coating Process
1. Mix Eudragit L-30D and Triethyl citrate, followed by addition of purified water. Mix the mixture thoroughly to produce solution B.
2. According to the same procedure as in the above 1-3 step, after the first coating of active ingredient coating is done, coat the second layer acid-resistant ingredient base coating using solution B.

III. Another Active Ingredient Fast Release Coating Process
1. Mix Povidone K-30, Talc, Lake into Ipecacuanha alkaloid and mix the mixture thoroughly to produce solution C.
2. After the second layer acid-resistant base coating is completed, use solution C to coat the active ingredient with the third layer another active ingredient fast release film coating.
3. Finally, allow the coating to dry for 10 minutes. The entire process is then complete.

IV. Pellet Capsule Filling Process.

Put the pellets with multi-layer coating active ingredient pellets into a pellet capsule filling machine to fill capsules.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A pharmaceutical composition with improved safety, comprising:
a selected amount of a vomit-inducing agent, wherein the selected amount is less than an amount needed to induce vomit in a user; and
a therapeutic agent.

2. The pharmaceutical composition of claim 1, wherein the therapeutic agent is selected from a sleeping drug, an anxiolytic, a hypnotic, and a contraceptive agent.

3. The pharmaceutical composition of claim 1, wherein the therapeutic agent is selected from diazepam, flunitrazepam, alprazolam, triazolam, fludiazepam, midazolam, estazolam, zopiclone, and a combination thereof.

4. The pharmaceutical composition of claim 1, wherein the vomit-inducing agent is one selected from emetine, cephaeline, and a combination thereof.

5. The pharmaceutical composition of claim 1, wherein the vomit-inducing agent is emetine, and the selected amount is in a range from about 0.2 mg to about 100 mg.

6. The pharmaceutical composition of claim 5, wherein the therapeutic agent is a sleeping drug, an anxiolytic agent, or a hypnotic agent, present in an amount ranging from about 0.02 mg to about 250 mg.

7. The pharmaceutical composition of claim 1, wherein the vomit-inducing agent is cephaeline, and the selected amount is in a range from about 0.2 mg to about 300 mg.

8. The pharmaceutical composition of claim 7, wherein the therapeutic agent is a sleeping drug, an anxiolytic agent, or a hypnotic agent, present in an amount ranging from about 0.02 mg to about 250 mg.

9. The pharmaceutical composition of claim 1, wherein the therapeutic agent is mifepristone.

10. The pharmaceutical composition of claim 9, wherein the vomit-inducing agent is selected from emetine, cephaeline, and a combination thereof.

11. The pharmaceutical composition of claim 9, wherein the vomit-inducing agent is emetine and the selected amount is in a range from about 0.25 mg to about 42 mg, and wherein mifepristone is present in an amount ranging from about 0.25 mg to about 150 mg.

12. The pharmaceutical composition of claim 11, wherein the selected amount of emetine is in a range from about 1.05 mg to 37.8 mg, and wherein mifepristone is present in an amount ranging from about 05 mg to 100 mg.

13. The pharmaceutical composition of claim 12, wherein the selected amount of emetine is in a range from about 2.1 mg to about 29.4 mg, and mifepristone is present in an amount ranging from 5 mg to 100 mg.

14. The pharmaceutical composition of claim 13, wherein the selected amount of emetine is in a range from about 4.2 mg to about 21 mg, and mifepristone is present in an amount ranging from 5 mg to 100 mg.

15. The pharmaceutical composition of claim 9, wherein the vomit-inducing agent is cephaeline and the selected amount is in a range from about 0.25 mg to about 126 mg, and wherein mifepristone is present in an amount ranging from about 0.25 mg to about 150 mg.

16. The pharmaceutical composition of claim 15, wherein the selected amount of cephaeline is in a range from about 1.05 mg to 114 mg, and wherein mifepristone is present in an amount ranging from about 05 mg to 100 mg.

17. The pharmaceutical composition of claim 1, wherein the pharmaceutical composition is in a form selected from a tablet, a capsule, a syrup, a solution, a suspension, and an elixir.

18. A composition with improved safety, comprising:
a selected amount of a vomit-inducing agent, wherein the selected amount is less than an amount needed to induce vomit in a user; and
a chemical selected from an agricultural chemical and a corrosive chemical.

19. The composition of claim 18, wherein the chemical is selected from parathion, parathion-methyl, folidol, methamidophos, mepanipyrim, mecarbam, sulfuric acid, hydrochloric acid.

20. The composition of claim 18, wherein the vomit-inducing agent is selected from emetine, cephaeline, and a combination thereof.

21. A method for preparing a pharmaceutical composition with improved safety according to claim 1, the method comprising:
preparing a core tablet of a therapeutic agent;
preparing a coating solution containing a selected amount of a vomit-inducing agent; and
coating the core tablet with the coating solution,
wherein the selected amount is such that a final amount of the vomit-inducing agent in the pharmaceutical composition is less than an amount needed to induce vomit in a user.

22. A method for preparing a pharmaceutical composition with improved safety according to claim 1, the method comprising:
preparing core pellets of a therapeutic agent;
preparing pellets containing a selected amount of a vomit-inducing agent; and
making capsules from the core pellets of the therapeutic agent and the pellets containing the vomit inducing agent,
wherein the selected amount is such that an amount of the vomit-inducing agent in each capsule is less than an amount needed to induce vomit in a user.
